(19) United States
(12) Patent Application Publication
(10) Pub. No.: US 2026/0007544 A1
(43) Pub. Date: Jan. 8, 2026

(54) APPARATUS AND METHOD FOR REMOVING AN INTERNAL LIMITING MEMBRANE OR EPIRETINAL MEMBRANE

(71) Applicant: KATALYST SURGICAL, LLC, Chesterfield, MO (US)

(72) Inventors: Carl C. AWH, Nashville, TN (US); Gregg D. SCHELLER, Wildwood, MO (US); Eric BASS, St. Louis, MO (US); Brad NIEHOFF, Lake St. Louis, MO (US)

(21) Appl. No.: 19/329,786

(22) Filed: Sep. 16, 2025

Related U.S. Application Data
(62) Division of application No. 17/013,059, filed on Sep. 4, 2020.
(60) Provisional application No. 62/896,262, filed on Sep. 5, 2019.

(51) Int. Cl. A61F 9/007; A61B 17/34

(52) U.S. Cl. CPC A61F 9/00736; A61B 17/3423

(57) ABSTRACT

An ophthalmic surgical instrument is provided that includes a handle having a handle distal end and a handle proximal end, a membrane removal instrument having an instrument proximal end attached to the handle distal end and an instrument distal end configured for engagement with an ophthalmic membrane, an aspiration port of the membrane removal instrument configured for engagement with at least a separated portion of the ophthalmic membrane with a suction force, and an aspiration lumen of the ophthalmic membrane removal instrument in operative communication with the aspiration port.

### CROSS-REFERENCE TO RELATED APPLICATIONS

This Non-Provisional Application claims priority to U.S. Provisional Application Ser. No. 62/896,262, filed on Sep. 5, 2019.

### STATEMENT REGARDING FEDERALLY SPONSORED RESEARCH

Not applicable.

### BACKGROUND

The present disclosure relates to a surgical instrument, and, more particularly, to an ophthalmic surgical instrument and method of use for removing an ophthalmic membrane, such as internal limiting membrane or an epiretinal membrane in an ophthalmic surgical procedure.

Abnormalities of the retina are often treated by surgical removal, or peeling, of membranes from the retina. An epiretinal membrane (ERM) is a layer of abnormal tissue that proliferates on either the inner or outer surface of the retina and can be a pathologic component in various conditions, such as, macular epiretinal membrane (“macular pucker”), proliferative vitreoretinopathy, and proliferative diabetic retinopathy. The internal limiting membrane (ILM) is a thin transparent membrane along the innermost surface of the normal retina. Peeling the ILM is a component of ophthalmic surgical procedures to treat retinal conditions including macular hole, macular ERM, diabetic macular edema, vitreomacular traction, proliferative diabetic retinopathy, retinal vein occlusion, myopic macular schisis, and proliferative vitreoretinopathy.

Generally, the surgical procedure involves a surgeon using a microsurgical instrument to grasp and peel the ophthalmic membrane. ILM or ERM, from the retina, referred to as membrane peeling. Membrane peeling typically involves the following steps: (1) staining or dying the ophthalmic membrane to improve visualization; (2) identification or creation of a membrane edge or flap sufficient to allow an instrument to achieve sufficient purchase or grasp to initiate peeling of the ophthalmic membrane from the retina; (3) peeling the ophthalmic membrane from the retina while maintaining sufficient purchase to keep contact with and control of the ophthalmic membrane; and (4) removal of the ophthalmic membrane from the eye. These steps can be accomplished using a variety of techniques, as in the following examples: (1) creation of a membrane edge or flap by using a relatively sharp instrument, such as a pick or barbed blade, or with forceps; (2) grasping the membrane flap with forceps and separating, such as by pulling, the ophthalmic membrane completely from the retina, or lifting the ophthalmic membrane with a pick but leaving a portion of the ophthalmic membrane attached to the retina; and (3) removing the forceps from the eye while maintaining grasp of the peeled ophthalmic membrane, or removing the pick and inserting a forceps to grasp and remove the elevated portion of the ophthalmic membrane. In order to remove the desired amount of ophthalmic membrane, any or all of these steps and techniques may need to be repeated multiple times, most commonly because the ophthalmic membrane may shred or tear as it is being peeled, requiring the surgeon to remove the ophthalmic membrane in separate pieces.

The ophthalmic membrane removal steps described above have associated complications and undesirable aspects. For example, creation of the membrane edge or flap with the forceps is technically challenging because the ILM is thin (approximately 4 microns) and “pinching” the ILM or ERM can result in damage to the underlying retina. Retinal damage may include nerve fiber layer defects and full-thickness retinal holes. In addition, repeated removal and insertion of the forceps from the eye is time-consuming and may increase the risk of peripheral retinal tears. Also, precisely positioning the forceps tips while simultaneously manually activating the forceps may cause fatigue of the surgeon and may lead to degradation of the surgeon's performance over time.

Accordingly, there is a need for a surgical device and method to initiate an edge or flap in the ophthalmic membrane without forceps and to remove the ophthalmic membrane with the same device, while minimizing the occurrences of removal and insertion of the device into the eye and eliminating manual activation of the device by the surgeon.

### BRIEF DESCRIPTION

In one embodiment, an ophthalmic surgical instrument is provided that includes a handle having a handle distal end and a handle proximal end, a membrane removal instrument having an instrument proximal end attached to the handle distal end and an instrument distal end configured for engagement with an ophthalmic membrane, an aspiration port of the membrane removal instrument configured for engagement with at least a separated portion of the ophthalmic membrane with a suction force, and an aspiration lumen of the ophthalmic membrane removal instrument in operative communication with the aspiration port. Optionally, the ophthalmic surgical instrument includes a spatula configured for separation of the ophthalmic membrane from a retina.

In another embodiment, an ophthalmic surgical instrument is provided that includes a membrane removal instrument having an instrument proximal end and an instrument distal end configured for engagement with a membrane, a connector of the membrane removal instrument configured for detachable connection to a handle, an aspiration port of the membrane removal instrument configured for engagement with at least a separated portion of the membrane with a suction force, and an aspiration lumen of the membrane removal instrument in operative communication with the aspiration port.

In another embodiment, a method of removing an ophthalmic membrane with a membrane removal instrument, is provided including inserting the membrane removal instrument into an eye, incising at least a portion of the ophthalmic membrane with a membrane removal instrument, engaging at least a portion of the ophthalmic membrane with an aspiration port of the membrane removal instrument with a suction force, separating at least a portion of the ophthalmic membrane from a retina with the membrane removal instrument, and removing at least a portion of the ophthalmic membrane from the eye through an aspiration lumen of the membrane removal instrument with the suction force, the aspiration lumen of the membrane being in operative communication with the aspiration port. Optionally, the method includes incising at least a portion of the ophthalmic membrane with a membrane removal instrument.

### BRIEF DESCRIPTION OF THE DRAWINGS

The present inventive subject matter will be better understood from reading the following description of non-limiting embodiments, with reference to the attached drawings, wherein below:

FIG. 1 is an exploded perspective view a surgical instrument;

FIG. 2A is a side view of the surgical instrument;

FIG. 2B is a cross-section view of the surgical instrument of FIG. 2A;

FIG. 3 is an enlarged perspective view of a membrane removal instrument;

FIG. 4A is an enlarged top view of the membrane removal instrument;

FIG. 4B is an enlarged side view of the membrane removal instrument;

FIG. 4C is an enlarged end view of the membrane removal instrument;

FIG. 5A is a cross-section view of the surgical instrument and an eye with a cannula;

FIG. 5B is a cross-section view of the surgical instrument engaged with the cannula and eye in a surgical procedure;

FIG. 6 is a perspective view of the membrane removal instrument engaged with an ophthalmic membrane;

FIG. 7 is another perspective view of the membrane removal instrument engaged with the ophthalmic membrane;

FIG. 8 is a perspective view of an aspiration port of the membrane removal instrument engaged with the ophthalmic membrane;

FIG. 9 is an alternate embodiment of the membrane removal instrument;

FIG. 10 is a second alternate embodiment of the membrane removal instrument;

FIG. 11 is a third alternate embodiment of the membrane removal instrument;

FIG. 12 is a fourth alternate embodiment of the membrane removal instrument;

FIG. 13 is a fifth alternate embodiment of the membrane removal instrument;

FIG. 14 is a sixth alternate embodiment of the membrane removal instrument;

FIG. 15 is a seventh alternate embodiment of the membrane removal instrument;

FIG. 16A is an enlarged top view of the seventh alternate embodiment of the membrane removal instrument;

FIG. 16B is an enlarged side view of the seventh alternate embodiment the membrane removal instrument; and

FIG. 16C is an enlarged end view of the seventh alternate embodiment of the membrane removal instrument.

Corresponding reference numerals indicate corresponding parts throughout the several figures of the drawings.

### DETAILED DESCRIPTION

The following detailed description illustrates the inventive subject matter by way of example and not by way of limitation. The description enables one of ordinary skill in the art to make and use the inventive subject matter, describes several embodiments of the inventive subject matter, as well as adaptations, variations, alternatives, and uses of the inventive subject matter. Additionally, it is to be understood that the inventive subject matter is not limited in its application to the details of construction and the arrangements of components set forth in the following description or illustrated in the drawings. The inventive subject matter is capable of other embodiments and of being practiced or being carried out in various ways. Also, it is to be understood that the phraseology and terminology used herein is for the purpose of description and should not be regarded as limiting on all embodiments of the inventive subject matter.

The terminology used herein is for the purpose of describing particular exemplary embodiments only and is not intended to be limiting. As used herein, the singular forms “a”, “an” and “the” may be intended to include the plural forms as well, unless the context clearly indicates otherwise. The terms “comprises.” “comprising,” “including,” and “having.” are inclusive and therefore specify the presence of stated features, integers, steps, operations, elements, and/or components, but do not preclude the presence or addition of one or more other features, integers, steps, operations, elements, components, and/or groups thereof. The steps, processes, and operations described herein are not to be construed as necessarily requiring their respective performance in the particular order discussed or illustrated, unless specifically identified as a preferred order of performance. It is also to be understood that additional or alternative steps may be employed.

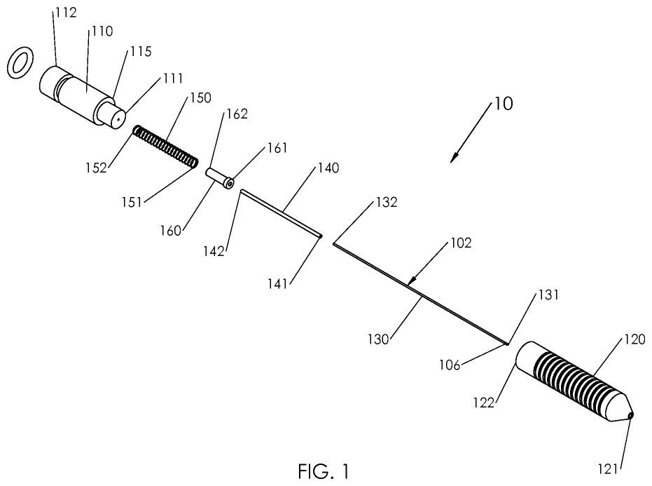

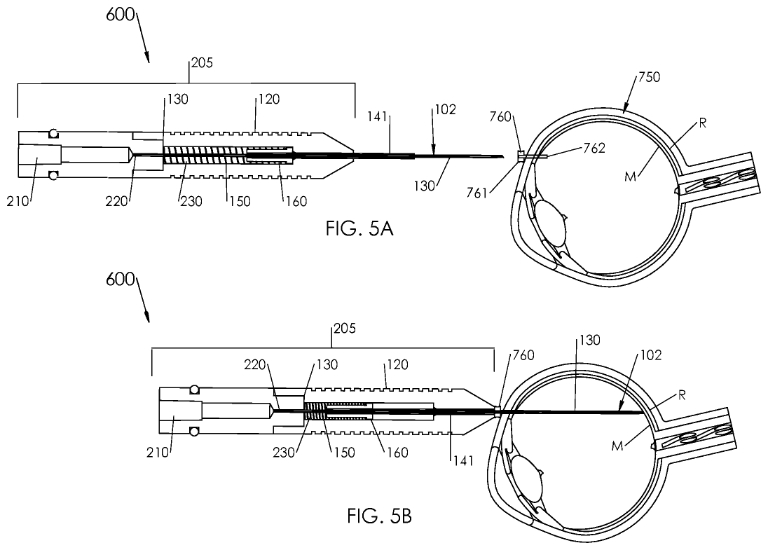

FIG. 1 is an exploded perspective view a surgical instrument 10. In one or more embodiments, a surgical instrument 10 may include a handle 100 configured for engagement by a surgeon and a membrane removal instrument 102 configured for insertion into an eye, and incision, engagement, and removal of an ophthalmic membrane M, including an internal limiting membrane (ILM) or epiretinal membrane (ERM), using a section pressure (See FIGS. 5-8).

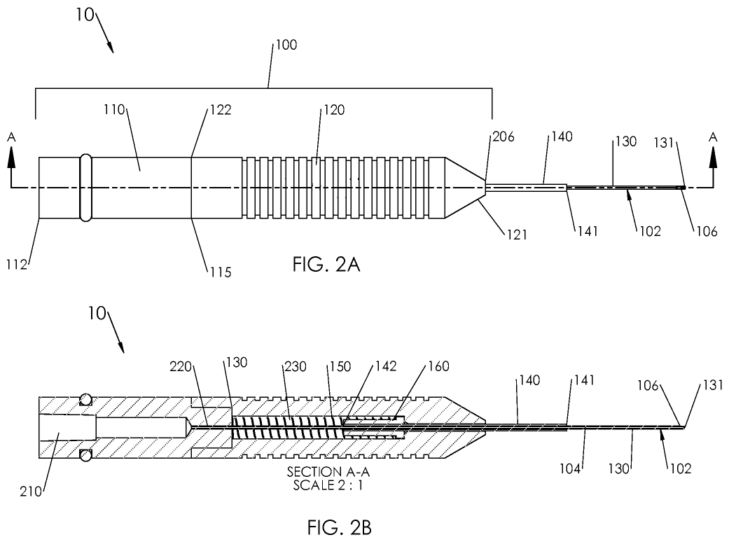

The membrane removal instrument 102 includes an inner instrument tube 130 having an inner instrument tube distal end 131, an inner instrument tube proximal end 132, and an aspiration lumen 104 extending therebetween along a longitudinal axis. The aspiration lumen 104 terminates at an aspiration port 106 proximate to the inner instrument tube distal end 131. As discussed in more detail below, inner instrument tube distal end 131, or membrane removal tip, of the membrane removal instrument 102 is configured to incise, engage, and remove the ophthalmic membrane M from the eye using suction pressure. The membrane removal instrument 102 is operatively connected to an aspiration supply (not shown) that provides the suction pressure. For example, a pump and tubing assembly (not shown) may attach to the handle 100 to communicate suction pressure through a proximal chamber 210, a lumen 220, a distal chamber 230, the aspiration lumen 104, and the aspiration port 106. (FIGS. 2A-2B).

The handle 100 includes a handle base component 110 having a handle base component distal end 111 and a handle base component proximal end 112, a handle base 120 having a handle base distal end 121 and a handle base proximal end 122, an inner instrument tube 130 having an inner instrument tube distal end 131 and an inner instrument tube proximal end 132, a protective sleeve 140 having a protective sleeve distal end 141 and a protective sleeve proximal end 142, a pressure mechanism 150 having a pressure mechanism distal end 151 and a pressure mechanism proximal end 152, and a piston 160 having a piston distal end 161 and a piston proximal end 162. Illustratively, handle base component 110 may comprise a handle base interface 115 configured to interface with handle base 120, e.g., at handle base proximal end 122. Handle base component 110, handle base 120, membrane removing instrument 130, protective sleeve 140, pressure mechanism 150, and piston 160 may be manufactured from any suitable material, e.g., polymers, metals, metal alloys, etc., or from any combination of suitable materials.

Although the embodiments of FIGS. 1-2B include a handle 100 with associated components, those skilled in the art will recognize that the surgical instrument 10 may include any suitable configuration of handle or other apparatus for a surgeon to engage and operate the membrane removal instrument 102.

Although the embodiments of FIGS. 1-2B include a protective sleeve 140 with associated components, those skilled in the art will recognize that other embodiments of the surgical instrument 10 may include any suitable configuration of protective sleeve, or may forgo having any protective sleeve or associated components.

FIG. 2A is a side view of the surgical instrument 10. FIG. 2B is a cross-section view of the surgical instrument of FIG. 2A. The handle 100 includes a handle distal end 206 and a handle proximal end 207. Illustratively, handle distal end 206 may comprise handle base distal end 121. In one or more embodiments, handle proximal end 207 may comprise handle base component proximal end 112. Illustratively, surgical instrument 10 may comprise a proximal chamber 210, a lumen 220, and a distal chamber 230. In one or more embodiments, a portion of protective sleeve 140 may be disposed within piston 160, e.g., protective sleeve proximal end 142 may be disposed within piston 160. Illustratively, a portion of protective sleeve 140 may be fixed to piston 160, e.g., protective sleeve proximal end 142 may be fixed to piston distal end 161. In one or more embodiments, protective sleeve 140 may be fixed to piston 160, e.g., by an adhesive or any other suitable fixation means. Illustratively, a portion of protective sleeve 140 may be fixed within an inner portion of piston 160, e.g., protective sleeve proximal end 142 may be fixed to an inner portion of piston 160. In one or more embodiments, protective sleeve 140 may be fixed within an inner portion of piston 160, e.g., by an adhesive or any other suitable fixation means.

Illustratively, a portion of inner instrument tube 130 may be fixed to a portion of handle base component 110, e.g., inner instrument tube proximal end 132 may be fixed to handle base component distal end 111. In one or more embodiments, inner instrument tube 130 may be fixed to handle base component 110. e.g., by an adhesive or any other suitable fixation means. Illustratively, a portion of inner instrument tube 130 may be disposed within handle base component 110, e.g., inner instrument tube proximal end 132 may be disposed within lumen 220. In one or more embodiments, inner instrument tube 130 may be fixed to an inner portion of handle base component 110, e.g., inner instrument tube proximal end 132 may be fixed within lumen 220. Illustratively, inner instrument tube 130 may be fixed to an inner portion of handle base component 110, e.g., by an adhesive or by any other suitable fixation means.

In one or more embodiments, handle base component 110 may be fixed to handle base 120. e.g., handle base interface 115 may be fixed to handle base proximal end 122. Illustratively, handle base component 110 may be fixed to handle base 120, e.g., by an adhesive or any other suitable fixation means. In one or more embodiments, inner instrument tube 130 may be disposed within lumen 220, distal chamber 230, piston 160, and protective sleeve 140. Illustratively, distal chamber 230 may comprise an inner bore of handle 205. In one or more embodiments, a portion of distal chamber 230 may be coated with a lubricant, e.g., Teflon. Illustratively, pressure mechanism 150 may be disposed within distal chamber 230. In one or more embodiments, piston 160 may be disposed within distal chamber 230. Illustratively, pressure mechanism 150 may be disposed between piston 160 and handle base component 110, e.g., pressure mechanism proximal end 152 may abut handle base component distal end 111 and pressure mechanism distal end 151 may abut piston proximal end 162.

In one or more embodiments, pressure mechanism 150 may be configured to provide a force. Illustratively, pressure mechanism 150 may be configured to provide a constant or uniform force. In one or more embodiments, pressure mechanism 150 may be configured to provide a variable force. Illustratively, pressure mechanism 150 may comprise a spring or a coil. In one or more embodiments, pressure mechanism 150 may comprise a spring having a spring constant in a range of 0.001 N/mm to 5.0 N/mm. Illustratively, pressure mechanism 150 may comprise a spring having a spring constant less than 0.001 N/mm or greater than 5.0 N/mm. In one or more embodiments, pressure mechanism 150 may comprise a pneumatic system. Illustratively, pressure mechanism 150 may be configured to provide a resistive force to resist an actuation of piston 160 within distal chamber 230. e.g., an actuation of piston 160 towards handle proximal end 207 and away from handle distal end 206. In one or more embodiments, pressure mechanism 150 may be configured to provide a facilitating force to facilitate an actuation of piston 160 within distal chamber 230. e.g., an actuation of piston 160 towards handle distal end 206 and away from handle proximal end 207.

In one or more embodiments, pressure mechanism 150 may be configured to provide a force, e.g., to piston proximal end 162. Illustratively, an application of a force to protective sleeve 140. e.g., applied to protective sleeve distal end 141, may be configured to actuate piston 160 within distal chamber 230, e.g., towards handle proximal end 207 and away from handle distal end 206. In one or more embodiments, an application of a force to protective sleeve distal end 141. e.g., a force having a magnitude greater than a force provided by pressure mechanism 150 to piston proximal end 162, may be configured to actuate piston 160 within distal chamber 230, e.g., towards handle proximal end 207 and away from handle distal end 206. Illustratively, an actuation of piston 160 within distal chamber 230, e.g., towards handle proximal end 207 and away from handle distal end 206, may be configured to retract protective sleeve 140 relative to inner instrument tube 130. In one or more embodiments, a retraction of protective sleeve 140 relative to inner instrument tube 130 may be configured to gradually expose a portion of inner instrument tube 130. Illustratively, pressure mechanism 150 may be configured to provide a force, e.g., a force to piston proximal end 162, configured to resist an actuation of piston 160 within distal chamber 230, e.g., towards handle proximal end 207 and away from handle distal end 206.

In one or more embodiments, pressure mechanism 150 may be configured to provide a force. e.g., to piston proximal end 162. Illustratively, a reduction of a force applied to protective sleeve 140, e.g., a force applied to protective sleeve distal end 141, may be configured to actuate piston 160 within distal chamber 230, e.g., towards handle distal end 206 and away from handle proximal end 207. In one or more embodiments, a reduction of a force applied to protective sleeve distal end 141. e.g., a reduction of a force having a magnitude greater than a force provided by pressure mechanism 150 to piston proximal end 162, may be configured to actuate piston 160 within distal chamber 230, e.g., towards handle distal end 206 and away from handle proximal end 207. Illustratively, an actuation of piston 160 within distal chamber 230, e.g., towards handle distal end 206 and away from handle proximal end 207, may be configured to extend protective sleeve 140 relative to inner instrument tube 130. In one or more embodiments, an extension of protective sleeve 140 relative to inner instrument tube 130 may be configured to gradually enclose a portion of inner instrument tube 130 within protective sleeve 140. Illustratively, pressure mechanism 150 may be configured to provide a force, e.g., a force to piston proximal end 162, configured to facilitate an actuation of piston160 within distal chamber 230. e.g., towards handle distal end 206 and away from handle proximal end 207.

In one or more embodiments, protective sleeve 140 may comprise a protective sleeve in a fully extended position 300, e.g., when piston 160 is fully extended within distal chamber 230. Illustratively, protective sleeve 140 may be configured to protect inner instrument tube 130, e.g., when protective sleeve 140 comprises a protective sleeve in a fully extended position 300.

In one or more embodiments, an application of a force to protective sleeve distal end 141 may be configured to retract protective sleeve 140 relative to inner instrument tube 130. Illustratively, an application of a force to protective sleeve distal end 141 may be configured to retract protective sleeve 140 from a protective sleeve in a fully extended position 300 to a protective sleeve in a first retracted position 310. In one or more embodiments, a retraction of protective sleeve 140 relative to inner instrument tube 130 may be configured to expose a portion of inner instrument tube 130, e.g., inner instrument tube distal end 131. Illustratively, a retraction of protective sleeve 140 relative to inner instrument tube 130 may be configured to retract piston 160 within distal chamber 230, e.g., actuate piston 160 within distal chamber 230 towards handle proximal end 207 and away from handle distal end 206. In one or more embodiments, a retraction of piston 160 within distal chamber 230 may be configured to retract a portion of protective sleeve 140 into distal chamber 230. e.g., protective sleeve proximal end 142. Illustratively, pressure mechanism 150 may be configured to provide a resistive force to resist a retraction of piston 160 within distal chamber 230. In one or more embodiments, protective sleeve distal end 141 may be retracted a first distance from inner instrument tube distal end 131, e.g., when protective sleeve 140 comprises a protective sleeve in a first retracted position 310. In one or more embodiments, an application of a force to protective sleeve distal end 141 may be configured to retract protective sleeve 140 relative to inner instrument tube 130. Illustratively, an application of a force to protective sleeve distal end 141 may be configured to retract protective sleeve 140 from a protective sleeve in a first retracted position 310 to a protective sleeve in a second retracted position 320. In one or more embodiments, a retraction of protective sleeve 140 relative to inner instrument tube 130 may be configured to expose a portion of inner instrument tube 130. Illustratively, a retraction of protective sleeve 140 relative to inner instrument tube 130 may be configured to retract piston 160 within distal chamber 230, e.g., actuate piston 160 within distal chamber 230 towards handle proximal end 207 and away from handle distal end 206. In one or more embodiments, a retraction of piston 160 within distal chamber 230 may be configured to retract a portion of protective sleeve 140 into distal chamber 230. Illustratively, pressure mechanism 150 may be configured to provide a resistive force to resist a retraction of piston 160 within distal chamber 230. In one or more embodiments, protective sleeve distal end 141 may be retracted a second distance from inner instrument tube distal end 131, e.g., when protective sleeve 140 comprises a protective sleeve in a second retracted position 320. Illustratively, the second distance from inner instrument tube distal end 131 may be any distance greater than the first distance from inner instrument tube distal end 131.

In one or more embodiments, an application of a force to protective sleeve distal end 141 may be configured to retract protective sleeve 140 relative to inner instrument tube 130. Illustratively, an application of a force to protective sleeve distal end 141 may be configured to retract protective sleeve 140 from a protective sleeve in a second retracted position 320 to a protective sleeve in a third retracted position 330. In one or more embodiments, a retraction of protective sleeve 140 relative to inner instrument tube 130 may be configured to expose a portion of inner instrument tube 130. Illustratively, a retraction of protective sleeve 140 relative to inner instrument tube 130 may be configured to retract piston 160 within distal chamber 230, e.g., actuate piston 160 within distal chamber 230 towards handle proximal end 207 and away from handle distal end 206. In one or more embodiments, a retraction of piston 160 within distal chamber 230 may be configured to retract a portion of protective sleeve 140 into distal chamber 230. Illustratively, pressure mechanism 150 may be configured to provide a resistive force to resist a retraction of piston 160 within distal chamber 230. In one or more embodiments, protective sleeve distal end 141 may be retracted a third distance from inner instrument tube distal end 131, e.g., when protective sleeve 140 comprises a protective sleeve in a third retracted position 330. Illustratively, the third distance from inner instrument tube distal end 131 may be any distance greater than the second distance from inner instrument tube distal end 131.

In one or more embodiments, protective sleeve 140 may comprise a protective sleeve in a fully retracted position 400, e.g., when piston 160 is fully retracted within distal chamber 230. Illustratively, protective sleeve 140 may be configured to expose inner instrument tube 130, e.g., when protective sleeve 140 comprises a protective sleeve in a fully retracted position 400.

In one or more embodiments, a reduction of a force applied to protective sleeve distal end 141 may be configured to extend protective sleeve 140 relative to inner instrument tube 130. Illustratively, a reduction of a force applied to protective sleeve distal end 141 may be configured to extend protective sleeve 140 from a protective sleeve in a fully retracted position 400 to a protective sleeve in a first extended position 410. In one or more embodiments, an extension of protective sleeve 140 relative to inner instrument tube 130 may be configured to cover a portion of inner instrument tube 130, e.g., an exposed portion of inner instrument tube 130. Illustratively, an extension of protective sleeve 140 relative to inner instrument tube 130 may be configured to extend piston 160 within distal chamber 230, e.g., actuate piston 160 within distal chamber 230 towards handle distal end 206 and away from handle proximal end 207. In one or more embodiments, an extension of piston 160 within distal chamber 230 may be configured to extend a portion of protective sleeve 140 out from distal chamber 230, e.g., protective sleeve distal end 141. Illustratively, pressure mechanism 150 may be configured to provide a facilitating force to facilitate an extension of piston 160 within distal chamber 230. In one or more embodiments, protective sleeve distal end 141 may be extended a first extended distance from handle distal end 206. e.g., when protective sleeve 140 comprises a protective sleeve in a first extended position 410.

In one or more embodiments, a reduction of a force applied to protective sleeve distal end 141 may be configured to extend protective sleeve 140 relative to inner instrument tube 130. Illustratively, a reduction of a force applied to protective sleeve distal end 141 may be configured to extend protective sleeve 140 from a protective sleeve in a first extended position 410 to a protective sleeve in a second extended position 420. In one or more embodiments, an extension of protective sleeve 140 relative to inner instrument tube 130 may be configured to cover a portion of inner instrument tube 130. e.g., an exposed portion of inner instrument tube 130. Illustratively, an extension of protective sleeve 140 relative to inner instrument tube 130 may be configured to extend piston 160 within distal chamber 230, e.g., actuate piston 160 within distal chamber 230 towards handle distal end 206 and away from handle proximal end 207. In one or more embodiments, an extension of piston 160 within distal chamber 230 may be configured to extend a portion of protective sleeve 140 out from distal chamber 230. Illustratively, pressure mechanism 150 may be configured to provide a facilitating force to facilitate an extension of piston 160 within distal chamber 230. In one or more embodiments, protective sleeve distal end 141 may be extended a second extended distance from handle distal end 206, e.g., when protective sleeve 140 comprises a protective sleeve in a second extended position 420. Illustratively, the second extended distance from handle distal end 206 may comprise any distance greater than the first extended distance from handle distal end 206.

In one or more embodiments, a reduction of a force applied to protective sleeve distal end 141 may be configured to extend protective sleeve 140 relative to inner instrument tube 130. Illustratively, a reduction of a force applied to protective sleeve distal end 141 may be configured to extend protective sleeve 140 from a protective sleeve in a second extended position 420 to a protective sleeve in a third extended position 430. In one or more embodiments, an extension of protective sleeve 140 relative to inner instrument tube 130 may be configured to cover a portion of inner instrument tube 130, e.g., an exposed portion of inner instrument tube 130. Illustratively, an extension of protective sleeve 140 relative to inner instrument tube 130 may be configured to extend piston 160 within distal chamber 230, e.g., actuate piston 160 within distal chamber 230 towards handle distal end 206 and away from handle proximal end 207. In one or more embodiments, an extension of piston 160 within distal chamber 230 may be configured to extend a portion of protective sleeve 140 out from distal chamber 230. Illustratively, pressure mechanism 150 may be configured to provide a facilitating force to facilitate an extension of piston 160 within distal chamber 230. In one or more embodiments, protective sleeve distal end 141 may be extended a third extended distance from handle distal end 206, e.g., when protective sleeve 140 comprises a protective sleeve in a third extended position 430. Illustratively, the third extended distance from handle distal end 206 may comprise any distance greater than the second extended distance from handle distal end 206.

In one or more embodiments, a surgeon may insert a surgical instrument tip, e.g., inner instrument tube distal end 131, into a cannula. A cannula is often used in surgical procedures having a surgical target site that is located beneath an outer layer of tissue. For example, an ophthalmic surgeon may use a cannula to perform a surgical procedure on a portion of an inner eye. Illustratively, an ophthalmic surgeon may make a small incision in an outer tissue of an eye, e.g., cornea, sclera, etc., and then the surgeon may insert a cannula into the small incision. Once the cannula is inserted into the small incision, the surgeon may utilize the cannula as a passageway to access a portion of the inner eye. e.g., a surgical target site. Illustratively, the surgeon may perform a surgical procedure by inserting a surgical instrument into the inner eye through the cannula, e.g., to perform a portion of the surgical procedure. After performing a portion of the surgical procedure, the surgeon may remove the surgical instrument from the inner eye through the cannula.

In one or more embodiments, protective sleeve 140 may be configured to protect inner instrument tube 130 as a portion of inner instrument tube, e.g., inner instrument tube distal end 131, is inserted into a cannula. Illustratively, inner instrument tube 130 may comprise an inner instrument tube diameter, a cannula may comprise a cannula diameter, and protective sleeve 140 may comprise a protective sleeve diameter. In one or more embodiments, an inner instrument tube diameter may be smaller than a cannula diameter. Illustratively, a cannula diameter may be smaller than or equal to a protective sleeve diameter. In one or more embodiments, a surgeon may guide protective sleeve distal end 141 towards an end of a cannula, e.g., when protective sleeve 140 comprises a protective sleeve in a fully extended position 300. Illustratively, the surgeon may insert inner instrument tube distal end 131 into the cannula and gradually extend inner instrument tube 130 through the cannula, e.g., to perform a portion of a surgical procedure. In one or more embodiments, as inner instrument tube 130 is gradually inserted into a cannula, protective sleeve 140 may be gradually extended over the cannula. Illustratively, as protective sleeve 140 is gradually extended over a cannula, protective sleeve distal end 141 may contact a barrier, e.g., a cannula flange, configured to prevent protective sleeve 140 from further extension over the cannula. In one or more embodiments, a surgeon may guide inner instrument tube 130 further into a cannula, e.g., to a surgical target site, after protective sleeve distal end 141 contacts a barrier. Illustratively, a further insertion of inner instrument tube 130 into a cannula after protective sleeve distal end 141 has contacted a barrier may be configured to retract protective sleeve 140 relative to inner instrument tube 130. In one or more embodiments, a further insertion of inner instrument tube 130 into a cannula after protective sleeve distal end 141 has contacted a barrier may be configured to retract protective sleeve 140 from a protective sleeve in a fully extended position 300 to a protective sleeve in a first retracted position 310.

Illustratively, protective sleeve 140 may be configured to protect inner instrument tube 130 as inner instrument tube 130 is removed from a cannula, e.g., after a surgeon performs a portion of a surgical procedure. In one or more embodiments, a surgeon may start to retract a portion of inner instrument tube 130 out from a cannula. e.g., when protective sleeve 140 comprises a protective sleeve in a fully retracted position 400. Illustratively, a retraction of inner instrument tube 130 out from a cannula may be configured to reduce a force applied to protective sleeve distal end 141. In one or more embodiments, a reduction of a force applied to protective sleeve distal end 141 may be configured to extend protective sleeve 140 relative to inner instrument tube 130. Illustratively, a reduction of a force applied to protective sleeve distal end 141 may be configured to extend protective sleeve 140 from a protective sleeve in a fully retracted position 400 to a protective sleeve in a first extended position 410. In one or more embodiments, an extension of protective sleeve 140 relative to inner instrument tube 130 may be configured to extend protective sleeve 140 over a portion of inner instrument tube 130, e.g., a portion of inner instrument tube 130 retracted out from a cannula.

Illustratively, one or more properties of a surgical instrument may be adjusted to attain one or more desired surgical instrument features. In one or more embodiments, a surgical instrument may be manufactured without pressure mechanism 150. Illustratively, pressure mechanism 150 may be disposed within distal chamber 230 wherein pressure mechanism proximal end 152 may abut piston distal end 161. In one or more embodiments, pressure mechanism 150 may be configured to provide a resistive force configured to resist an extension of protective sleeve 140 relative to inner instrument tube 130. Illustratively, pressure mechanism 150 may be configured to provide a facilitating force configured to facilitate a retraction of protective sleeve 140 relative to inner instrument tube 130.

For example, protective sleeve 140 may be configured to retract relative to inner instrument tube 130, e.g., to expose inner instrument tube distal end 131 for a surgical procedure. Illustratively, protective sleeve 140 may be configured to retract relative to inner instrument tube 130, e.g., but protective sleeve 140 may not be configured to extend relative to inner instrument tube 130. For example, protective sleeve 140 may be configured to extend relative to inner instrument tube 130, e.g., to enclose inner instrument tube distal end 131 within protective sleeve 140 after a surgical procedure. In one or more embodiments, protective sleeve 140 may be configured to extend relative to inner instrument tube 130, e.g., but protective sleeve 140 may not be configured to retract relative to inner instrument tube 130.

Illustratively, a switch or a lever may be added to manipulate a retraction or an extension of protective sleeve 140 relative to inner instrument tube 130. In one or more embodiments, either an actuation of a switch or an application of a force to protective sleeve distal end 141 may be configured to retract protective sleeve 140 relative to inner instrument tube 130. Illustratively, either an actuation of a lever or an application of a force to protective sleeve distal end 141 may be configured to retract protective sleeve 140 relative to inner instrument tube 130. In one or more embodiments, either an actuation of a switch or a reduction of a force applied to protective sleeve distal end 141 may be configured to extend protective sleeve 140 relative to inner instrument tube 130. Illustratively, either an actuation of a lever or a reduction of a force applied to protective sleeve distal end 141 may be configured to extend protective sleeve 140 relative to inner instrument tube 130. In one or more embodiments, either an actuation of a switch or an actuation of a lever may be configured to retract protective sleeve 140 relative to inner instrument tube 130. Illustratively, either an actuation of a switch or an actuation of a lever may be configured to extend protective sleeve 140 relative to inner instrument tube 130.

In one or more embodiments, inner instrument tube 130 may be configured to house a surgical instrument tip. Illustratively, inner instrument tube 130 may comprise a surgical instrument tip. In one or more embodiments, a retraction of protective sleeve 140 relative to inner instrument tube 130 may be configured to expose a surgical instrument tip, e.g., before a surgeon performs a surgical procedure. Illustratively, an extension of protective sleeve 140 relative to inner instrument tube 130 may be configured to protect a surgical instrument tip, e.g., before or after a surgeon performs a surgical procedure. In one or more embodiments, one or more properties of pressure mechanism 150 may be adjusted, e.g., to vary an amount of force applied to protective sleeve distal end 141 configured to retract protective sleeve 140 relative to inner instrument tube 130. Illustratively, a spring constant of pressure mechanism 150 may be adjusted to vary an amount of force applied to protective sleeve distal end 141 configured to retract protective sleeve 140 relative to inner instrument tube 130.

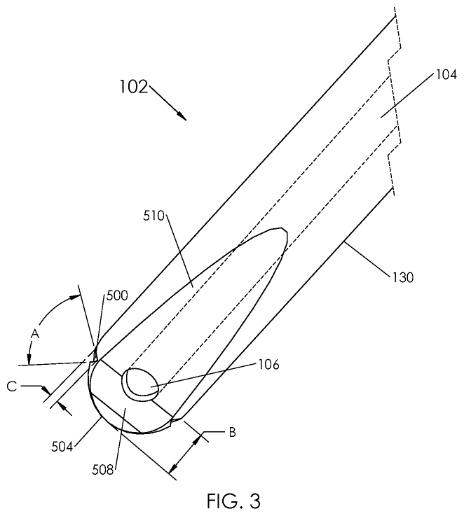

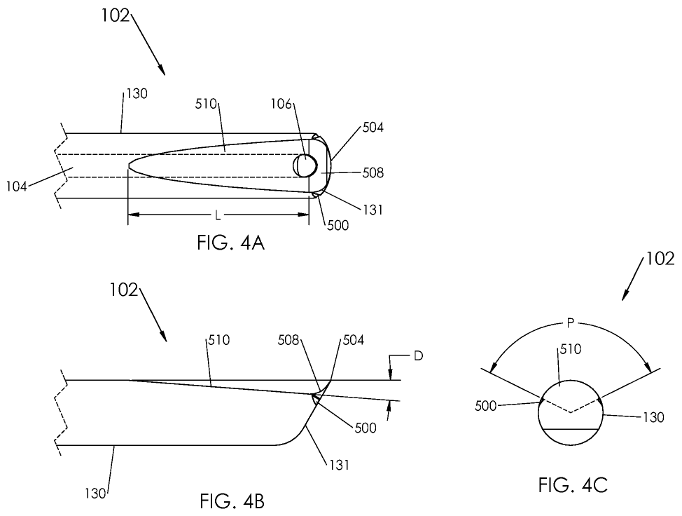

FIG. 3 is an enlarged perspective view the membrane removal instrument. FIG. 4A is an enlarged top view of the membrane removal instrument. FIG. 4B is an enlarged side view of the membrane removal instrument. FIG. 4C is an enlarged end view of the membrane removal instrument. As previously discussed above, the membrane removal instrument 102 includes the inner instrument tube 130 having an inner instrument tube distal end 131, an inner instrument tube proximal end 132, and an aspiration lumen 104 extending therebetween along a longitudinal axis. The aspiration lumen 104 terminates at the aspiration port 106 proximate to the inner instrument tube distal end 131.

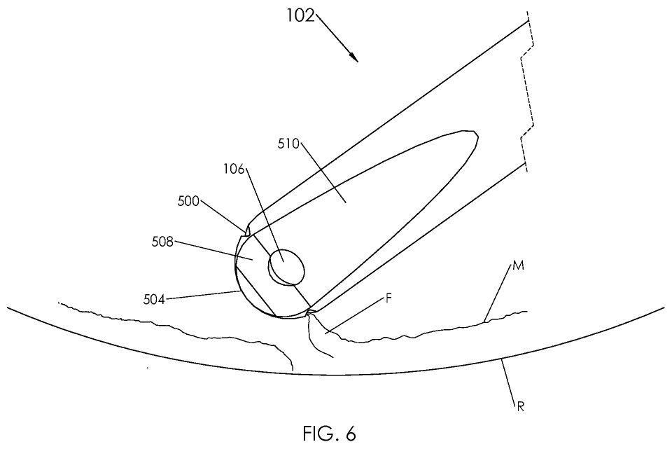

The membrane removal instrument 102 includes a pair of micro-picks 500 that are configured to engage and incise the ophthalmic membrane M to form a membrane flap F (FIG. 6). For example, the micro-picks 500 are generally V-shaped notches having a predetermined angle A and a depth C. In addition, the micro-picks 500 may be separated from each other about a central axis at a separation angle P and positioned at a length B from a tip 504 of the instrument 102. In the embodiment of FIG. 3, the micro-picks 500 preferably have an angle A of about 35°-65°, a depth C of about 0.001-0.002″, are positioned from each other at separation angle P of about 80°-110°, and are positioned at a length B of about 0.005-0.006″ from the tip 504. However, those skilled in the art will recognize that the micro-picks may include any suitable angle, depth, and separation angle. In addition, the membrane removal instrument 102 may includes any number of micro-picks 500, including one, positioned any separation angle. In alternate embodiments, the micro-picks may also comprise protrusions from the membrane removal instrument 102 of a predetermined size and shape.

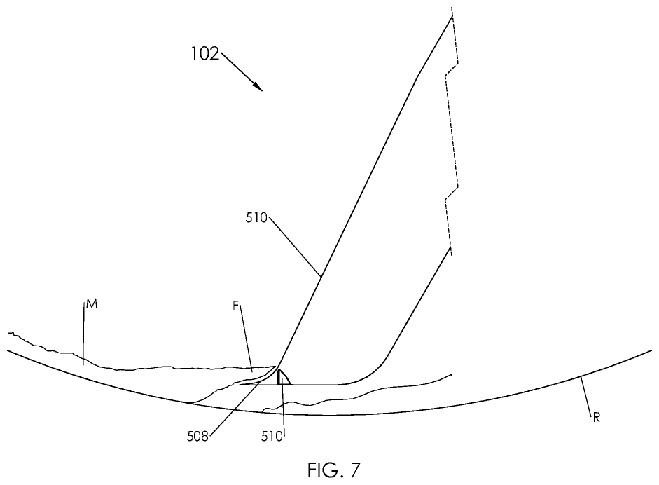

The membrane removal instrument 102 includes a spatula 508 proximate the inner instrument distal end 131 that is configured to initiate separation of the ophthalmic membrane M from the retina. For example, the spatula 508 may be an arcuate concave surface that terminates in an edge 504. The edge 504 should not be sharp, but rather be smooth enough to prevent any damage to the ophthalmic membrane M during engagement. The spatula 508 includes a tapered section 510 that extends rearwardly for a length L, preferably about 0.0625″. The tapered section 210 has a taper with an angle D, preferably about 1°-10°, relative to the longitudinal axis of the membrane removal instrument 102. The aspiration port 106 is positioned along the surface of the tapered section 510 and/or spatula 508. The aspiration port 106 is preferably a circular hole having a diameter of about 0.007″, however any suitable size and shape can be used. Although the inner instrument tube 130 is shown as a cylindrical tube with a circular cross-section, such as a 25-gauge hypodermic tube, the inner instrument tube 130 can be any suitable size, gauge, and shape, including having an oval, square, or other shape cross-section. As shown in FIG. 7, in operation, the spatula 508 may be inserted between the ophthalmic membrane and the retina to promote separation and allow for the aspiration port 106 to engage the ophthalmic membrane M with suction pressure.

The membrane removal instrument 102 may be manufactured using any suitable process or method, including, but not limited to, electric discharge manufacturing (EDM), additive manufacturing, casting, forging, molding, machining, forming, and the like. The membrane removal instrument 102 may comprise any suitable material including metal, polymers, 3D printing materials, ceramics, elastomers, and the like.

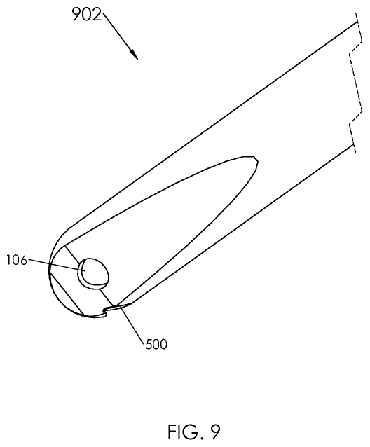

FIG. 9 is an alternate embodiment of the instrument tip. In an exemplary embodiment, the membrane removal instrument 902 is identical to the embodiment of FIG. 3 except for the configuration of the micro-picks 500. As shown in FIG. 9, the membrane removal instrument 102 includes only a single micro-pick 500. Optionally, the micro-pick 500 includes an angle of about 60°-90°, a depth of about 0.001-0.002″, a length of about 0.003″, and are positioned at a length of about 0.004″ from the tip 504.

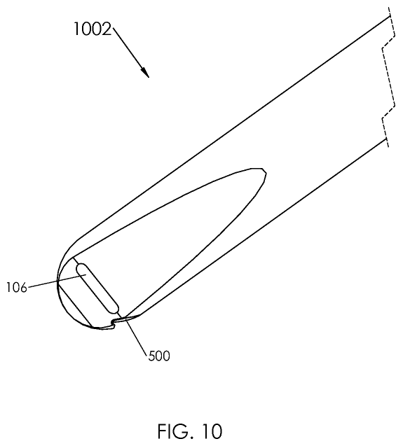

FIG. 10 is a second alternate embodiment of the membrane removal instrument 102. In an exemplary embodiment, the membrane removal instrument 1002 is identical to the embodiment of FIG. 9 except for the configuration of the aspiration port. As shown in FIG. 10, the aspiration port 106 is a slot having a length of about 0.010″ and a height of about 0.004″.

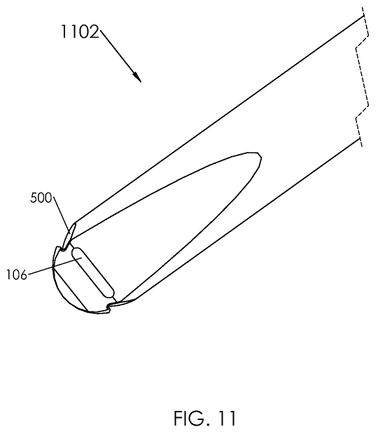

FIG. 11 is a third alternate embodiment of the membrane removal instrument 1102. In an exemplary embodiment, the membrane removal instrument 1102 is identical to the embodiment of FIG. 10 except for the configuration of the micro-picks. As shown in FIG. 11, the membrane removal instrument 102 includes a pair of micro-picks 500 separated from each other about a central axis at a separation angle of about 80°-110°.

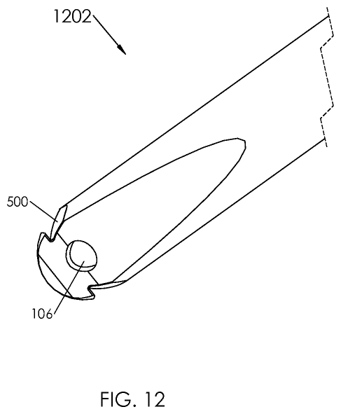

FIG. 12 is a fourth alternate embodiment of the membrane removal instrument 1202. In an exemplary embodiment, the membrane removal instrument 1202 is identical to the embodiment of FIG. 3 except for the configuration of the micro-picks 500. As shown in FIG. 12, the membrane removal instrument 102 includes micro-picks 500 includes an angle of about 45°-80°, a depth of about 0.003-0.004″, a length of about 0.003″-0.004″, and are positioned at a length of about 0.003″ from the tip 504.

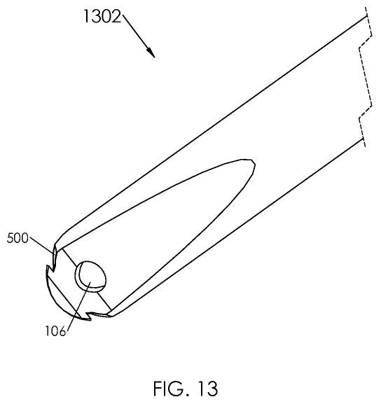

FIG. 13 is a fifth alternate embodiment of the membrane removal instrument 1302. In an exemplary embodiment, the membrane removal instrument 1302 is identical to the embodiment of FIG. 3 except for the configuration of the micro-picks 500. As shown in FIG. 13, the membrane removal instrument 102 includes micro-picks 500 includes an angle of about 35°-65°, a depth of about 0.003-0.004″, a length of about 0.003″, and are positioned at a length of about 0.002-0.003″ from the tip 504.

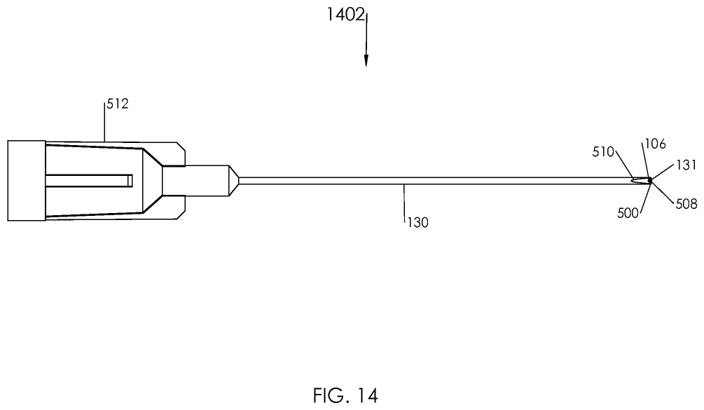

FIG. 14 is a sixth alternate embodiment of the membrane removal instrument. In an exemplary embodiment, the membrane removal instrument 1402 may provided without a handle 100. For example, the membrane removal instrument 1402 may include a connector 512 proximate the proximal end 132 of the instrument 102. The connector 512 may be configured for detachable connection with a handle or other suitable device. The connector 512 may include any suitable connection configuration, including, but not limited to quick-connect, luer hub, female hub, male hub, luer lock, and the like. In the embodiment shown in FIG. 14, the membrane removal instrument 1402 is identical to the embodiment of FIG. 6 with the exception of the connector 512. However, those skilled in the are will recognize that the connector 512 can be used with any other embodiment of the membrane removal instrument, including the embodiments of FIGS. 9-13.

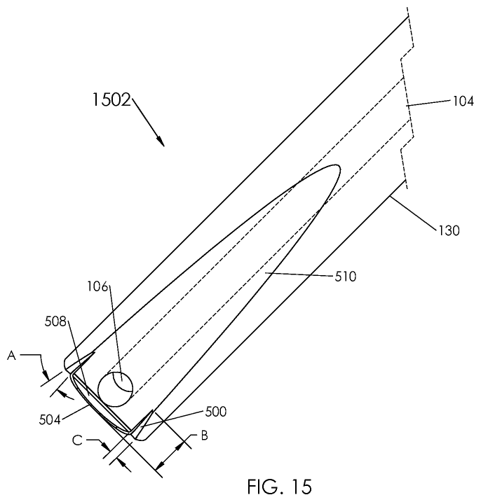

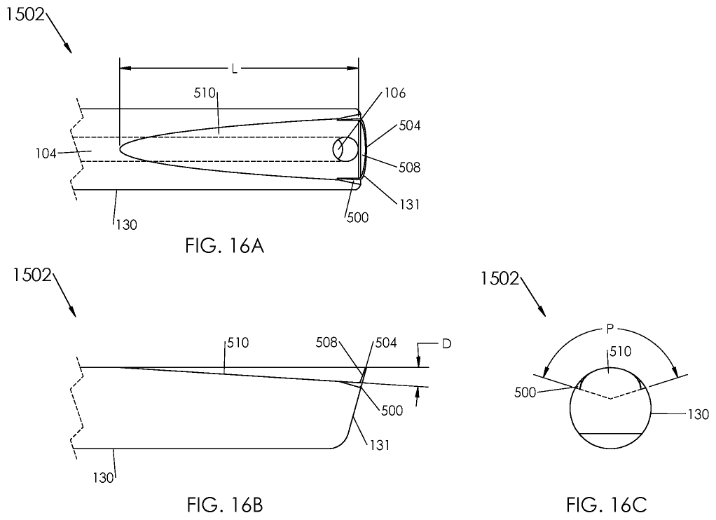

FIG. 15 is a seventh alternate embodiment of the membrane removal instrument 1502. FIG. 16A is an enlarged top view of the seventh alternate embodiment of the membrane removal instrument 1502. FIG. 16B is an enlarged side view of the seventh alternate embodiment the membrane removal instrument 1502. FIG. 16C is an enlarged end view of the seventh alternate embodiment of the membrane removal instrument 1502. In an exemplary embodiment, the membrane removal instrument 1502 is similar to the membrane removal instrument 102 of FIGS. 3-4C, and includes the inner instrument tube 130 having an inner instrument tube distal end 131, an inner instrument tube proximal end 132, and an aspiration lumen 104 extending therebetween along a longitudinal axis. The aspiration lumen 104 terminates at the aspiration port 106 proximate to the inner instrument tube distal end 131.

The membrane removal instrument 102 includes a pair of micro-picks 500 that are configured to engage and incise the ophthalmic membrane M to form a membrane flap F (FIG. 6). For example, the micro-picks 500 are generally V-shaped notches having a predetermined angle A and a depth C. In addition, the micro-picks 500 may be separated from each other about a central axis at a separation angle P and positioned at a length B from a tip 504 of the instrument 102. In the embodiment of FIG. 15, the micro-picks 500 preferably have an angle A of about 65°-75°, a depth C of about 0.0010-0.0020″, are positioned from each other at separation angle P of about 109°-119°, and are positioned at a length of about 0.007-0.013″ from the tip 504. However, those skilled in the art will recognize that the micro-picks may include any suitable angle, depth, and separation angle. In addition, the membrane removal instrument 102 may includes any number of micro-picks 500, including one, positioned any separation angle. In alternate embodiments, the micro-picks may also comprise protrusions from the membrane removal instrument 102 of a predetermined size and shape.

The membrane removal instrument 102 includes a spatula 508 proximate the inner instrument distal end 131 that is configured to initiate separation of the ophthalmic membrane M from the retina. For example, the spatula 508 may be an arcuate concave surface that terminates in an edge 504. The edge 504 should not be sharp, but rather be smooth enough to prevent any damage to the ophthalmic membrane M during engagement. The spatula 508 includes a tapered section 510 that extends rearwardly for a length L, preferably about 0.063″. The tapered section 210 has a taper with an angle D, preferably about 2.9°-4.6°, relative to the longitudinal axis of the membrane removal instrument 102. The aspiration port 106 is positioned along the surface of the tapered section 510 and/or spatula 508. The aspiration port 106 is preferably a circular hole having a diameter of about 0.0055-0.0065″, however any suitable size and shape can be used. Although the inner instrument tube 130 is shown as a cylindrical tube with a circular cross-section, such as a 25-gauge hypodermic tube, the inner instrument tube 130 can be any suitable size, gauge, and shape, including having an oval, square, or other shape cross-section. As shown in FIG. 7, in operation, the spatula 508 may be inserted between the ophthalmic membrane and the retina to promote separation and allow for the aspiration port 106 to engage the ophthalmic membrane M with suction pressure.

FIG. 5A is a cross-section view of the surgical instrument and an eye with a cannula. FIG. 5B is a cross-section view of the surgical instrument engaged with the cannula and eye in a surgical procedure. In one or more embodiments, a surgical instrument 600 may comprise a surgical instrument in a first surgical position 700 when support sleeve 540 is fully extended relative to inner instrument tube 130. Illustratively, a surgical instrument 600 may comprise a surgical instrument in a first surgical position 700 when support sleeve distal end 541 is aligned with a cannula 760 inserted in an eye 750. In one or more embodiments, cannula 760 may comprise a support sleeve interface 761 and a cannula end 762. An ophthalmologic surgeon may insert cannula 760 into an incision in corneal or scleral tissue 751 of eye 750, e.g., in order to access a surgical target site within inner eye 752. After cannula 760 is inserted into eye 750, the ophthalmologic surgeon may access inner eye 752 via cannula 760 without causing excessive trauma to corneal or scleral tissue 751.

In one or more embodiments, a surgical instrument 600 may comprise a surgical instrument in a second surgical position 710 when support sleeve 540 is partially retracted relative to inner instrument tube 130. Illustratively, a surgical instrument 600 may comprise a surgical instrument in a second surgical position 710 when support sleeve distal end 541 is interfaced with support sleeve interface 761. In one or more embodiments, a surgeon may guide a surgical instrument 600 from a surgical instrument in a first position 700 to a surgical instrument in a second position 710 by advancing support sleeve distal end 541 towards support sleeve interface 761 until support sleeve distal end 541 contacts support sleeve interface 761, and then guiding inner instrument tube distal end 131 through cannula 760, e.g., and into inner eye 752. Illustratively, when support sleeve distal end 541 is interfaced with support sleeve interface 761, the surgeon may guide inner instrument tube 130 through cannula 760 by advancing handle distal end 206 towards cannula 760.

In one or more embodiments, a diameter of inner instrument tube 130 may be less than a diameter of cannula 760. Illustratively, a diameter of support sleeve 540 may be greater than or equal to a diameter of cannula 760. In one or more embodiments, inner instrument tube 130 may be manufactured with dimensions configured to allow inner instrument tube 130 to be inserted through cannula 760. Illustratively, support sleeve 540 may be manufactured with dimensions configured to prevent support sleeve 540 from being inserted through cannula 760. In one or more embodiments, advancing handle distal end 206 towards cannula 760 after support sleeve distal end 541 has contacted support sleeve interface 761 may be configured to apply a force to support sleeve distal end 541. Illustratively, an application of a force to support sleeve distal end 541 may be configured to retract support sleeve 540 relative to inner instrument tube. In one or more embodiments, as inner instrument tube 130 is gradually advanced through cannula 760, support sleeve 540 may be gradually retracted into handle 205, e.g., into distal chamber 230. Illustratively, pressure mechanism 150 may be configured to provide a resistive force to resist a retraction of support sleeve 540 relative to inner instrument tube 130.

Illustratively, a surgical instrument 600 may comprise a surgical instrument in a third surgical position 720 when support sleeve 540 is partially retracted relative to inner instrument tube 130. In one or more embodiments, a surgical instrument 600 may comprise a surgical instrument in a third surgical position 720 when a surgeon has advanced inner instrument tube distal end 131 a particular distance within inner eye 752, e.g., the particular distance within inner eye 752 may be associated with a surgical target site location. Illustratively, a surgeon may guide a surgical instrument 600 from a surgical instrument in a second position 710 to a surgical instrument in a third position 720 by advancing handle distal end 206 towards cannula 760. In one or more embodiments, advancing handle distal end 206 towards cannula 760 may be configured to retract support sleeve 540 relative to inner instrument tube 130. Illustratively, pressure mechanism 150 may be configured to provide a resistive force to resist a retraction of support sleeve 540 relative to inner instrument tube 130.

In one or more embodiments, a portion of support sleeve 540 may be configured to enclose a portion of inner instrument tube 130, e.g., to increase a stiffness of a portion of inner instrument tube 130. Illustratively, a portion of support sleeve 540 may be configured to enclose a portion of inner instrument tube 130, e.g., to decrease a flexibility of a portion of inner instrument tube 130. In one or more embodiments, a surgeon may attempt to adjust a position of inner instrument tube 130 within inner eye 752, e.g., by adjusting a position of handle 205. Illustratively, a portion of support sleeve 540 may be configured to prevent inner instrument tube 130 from actuating unexpectedly within inner eye 752, e.g., in response to an adjustment of a position of handle 205. In one or more embodiments, a portion of support sleeve 540 may be configured to prevent inner instrument tube 130 from bending or flexing, e.g., in response to an adjustment of a position of handle 205.

In one or more embodiments, one or more properties of a surgical instrument may be adjusted to attain one or more surgical instrument features. Illustratively, support sleeve 540 may be replaced with a wire configured to increase a stiffness of a portion of inner instrument tube 130. In one or more embodiments, a surgical instrument may not comprise a support sleeve 540. For example, a portion of inner instrument tube 130 may be manufactured with a material configured to increase a stiffness of the portion of inner instrument tube 130. Illustratively, a portion of inner instrument tube 130 may be manufactured with dimensions configured to increase a stiffness of the portion of inner instrument tube 130. In one or more embodiments, a surgical instrument may comprise multiple layers of sleeves. Illustratively, a protective sleeve 140 may be disposed over support sleeve 540, and support sleeve 540 may be disposed over inner instrument tube 130. In one or more embodiments, protective sleeve 140 may be configured to protect support sleeve 540 and inner instrument tube 130 from damage before or after a surgical procedure.

Illustratively, pressure mechanism 150 may be configured to minimize a force applied to eye 750 during a surgical procedure. In one or more embodiments, pressure mechanism 150 may comprise a spring having a spring constant in a range of 0.0001 N/m to 100 N/m, e.g., to minimize a force applied to eye 750 during a surgical procedure. Illustratively, pressure mechanism 150 may comprise a spring having a spring constant below 0.0001 N/m or above 100 N/m, e.g., to minimize a force applied to eye 750 during a surgical procedure. In one or more embodiments, support sleeve 540 may be configured to minimize a force applied to eye 750 during a surgical procedure. Illustratively, a geometry of support sleeve 540 may be configured to dissipate a force, e.g., a force provided by pressure mechanism 150, to minimize a force applied to eye 750 during a surgical procedure. For example, support sleeve 540 may be manufactured with a diamond or a triangle mesh pattern to dissipate a force provided by pressure mechanism 150, e.g., to minimize a force applied to eye 750.

In one or more embodiments, support sleeve distal end 541 may be configured to minimize a force applied to eye 750 during a surgical procedure, e.g., by dissipating a force provided by pressure mechanism 150. Illustratively, support sleeve distal end 541 may comprise a cushion material or a padding material configured to dissipate a force provided by pressure mechanism 150, e.g., to minimize a force applied to eye 750 during a surgical procedure. In one or more embodiments, a surface area of support sleeve distal end 541 may be increased to transfer a force provided by pressure mechanism 150 over a larger area of eye 750, e.g., to minimize a risk of trauma to eye 750. Illustratively, a surface area of support sleeve distal end 541 may be decreased to transfer a force provided by pressure mechanism 150 over a smaller area of eye 750, e.g., to minimize an area of eye 750 exposed to a force provided by pressure mechanism 150.

In one or more embodiments, one or more properties of a surgical instrument may be configured to minimize an increase in intraocular pressure (“IOP”) during a surgical procedure. The medically accepted normal range of IOP is between 10 mmHg and 20 mmHg. Normal IOP may increase or decrease approximately 3 mmHg as a result of normal conditions. Illustratively, pressure mechanism 150 may be configured to provide a force having a magnitude configured to increase IOP by less than 3 mmHg during a surgical procedure. In one or more embodiments, support sleeve 540 may be configured to dissipate a force provided by pressure mechanism 150 wherein the dissipated force is configured to increase IOP by less than 3 mmHg during a surgical procedure.

Illustratively, a surgical instrument may be configured to both protect inner instrument tube 130 from damage before or after a surgical procedure and to increase a stiffness of a portion of inner instrument tube 130, e.g., to prevent inner instrument tube 130 from actuating in an unexpected manner when inserted in a cannula. In one or more embodiments, protective sleeve 140 may be configured with the functionality of support sleeve 540. Illustratively, support sleeve 540 may be configured with the functionality of protective sleeve 140. Accordingly, support sleeve 540 and protective sleeve 140 may be considered as interchangeable in any one or more embodiments.

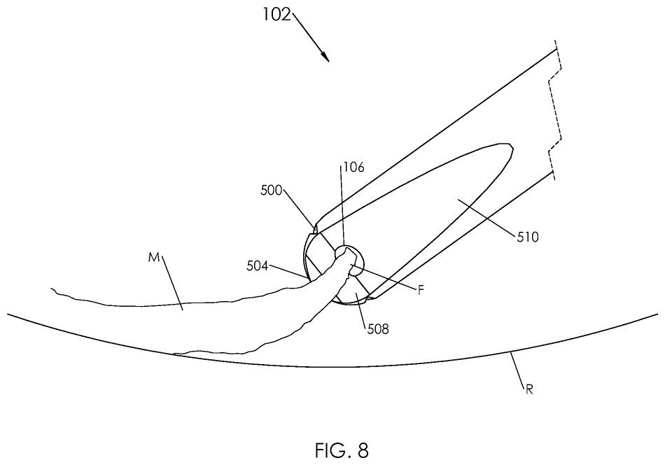

FIG. 6 is a perspective view of the membrane removal instrument engaged with the ophthalmic membrane. FIG. 7 is another perspective view of the membrane removal instrument engaged with the ophthalmic membrane. FIG. 8 is a perspective view of an aspiration port of the membrane removal instrument engaged with the ophthalmic membrane. In operation, during a membrane peeling surgical procedure the membrane removal instrument 102 inserts through the cannula 760 into the eye 750 and is positioned proximate the ophthalmic membrane M. At least one of the micro-picks 500 engages at least a portion of the ophthalmic membrane M to form a membrane flap F that can be engaged by the membrane removal instrument 102. For example, the micro-pick 500 incises the ophthalmic membrane M without causing any damage to the underlying retina R. The spatula 508 inserts between the ophthalmic membrane M and the retina R to promote separation and allow for the aspiration port 106 to engage the ophthalmic membrane M with suction force. The spatula 508 may be inserted with the aspiration port 106 positioned away from the retina R, so that the retina R is not affected by the suction force. The aspiration port 106 engages the membrane flap F of ophthalmic membrane M with suction force so that the surgeon can separate the ophthalmic membrane M from the retina M by pulling the microsurgical removal instrument 102 and the engaged membrane flap F away from the retina R. Optionally, the ophthalmic membrane M is removed in a minimum number of portions, including one. However, if needed, the aspiration port 106 can engage and remove the ophthalmic membrane M in multiple pieces. Once removed, the separated ophthalmic membrane M is aspirated through the aspiration port 106, through the aspiration lumen 104 to a disposal location (not shown). The communication or aspiration of the separated ophthalmic membrane M through the aspiration port 106 and aspiration lumen 104 may occur while the microsurgical removal instrument 102 remains inserted within the eye. In this way, the microsurgical removal instrument 102 does not need to be removed and/or reinserted during the membrane peeling surgical procedure.

Although the embodiments of the surgical instrument 10 and method of use described above relate to the removal of ophthalmic membranes, those skilled in the art will recognize that the surgical instrument 10 may be used in other surgical procedures. For example, the surgical instrument may be used to create an anterior capsule opening for a cataract surgical procedure.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the subject matter set forth herein without departing from its scope. While the dimensions and types of materials described herein are intended to define the parameters of the disclosed subject matter, they are by no means limiting and are exemplary embodiments. Many other embodiments will be apparent to those of skill in the art upon reviewing the above description. The scope of the subject matter described herein should, therefore, be determined with reference to the appended claims, along with the full scope of equivalents to which such claims are entitled. In the appended claims, the terms “including” and “in which” are used as the plain English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112 (f), unless and until such claim limitations expressly use the phrase “means for” followed by a statement of function void of further structure.

As used herein, an element or step recited in the singular and proceeded with the word “a” or “an” should be understood as not excluding plural of said elements or steps, unless such exclusion is explicitly stated. Furthermore, references to “one embodiment” of the presently described subject matter are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

This written description uses examples to disclose several embodiments of the subject matter set forth herein, including the best mode, and also to enable a person of ordinary skill in the art to practice the embodiments of disclosed subject matter, including making and using the devices or systems and performing the methods. The patentable scope of the subject matter described herein is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

The foregoing description of certain embodiments of the present inventive subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, communication unit, control system, etc.) may be implemented in a single piece of hardware (for example, a general-purpose signal processor, microcontroller, random access memory, hard disk, and the like). Similarly, the programs may be stand-alone programs, may be incorporated as subroutines in an operating system, may be functions in an installed software package, and the like. The various embodiments are not limited to the arrangements and instrumentality shown in the drawings.

Since certain changes may be made in the above-described systems and methods, without departing from the spirit and scope of the inventive subject matter herein involved, it is intended that all of the subject matter of the above description or shown in the accompanying drawings shall be interpreted merely as examples illustrating the inventive concept herein and shall not be construed as limiting the inventive subject matter.

Changes can be made in the above constructions without departing from the scope of the disclosure, it is intended that all matter contained in the above description or shown in the accompanying drawings shall be interpreted as illustrative and not in a limiting sense.

What is claimed is:

1. An ophthalmic surgical instrument, comprising:
a membrane removal instrument having an instrument proximal end and an instrument distal end configured for engagement with an ophthalmic membrane;
a connector of the membrane removal instrument configured for detachable connection to a handle;
an aspiration port of the membrane removal instrument configured for engagement with at least a separated portion of the ophthalmic membrane with a suction force; and
an aspiration lumen of the membrane removal instrument in operative communication with the aspiration port.

2. The ophthalmic surgical instrument of claim 1, wherein the membrane removal instrument includes at least one micro-pick configured for engagement with an ophthalmic membrane.

3. The ophthalmic surgical instrument of claim 1, wherein the membrane removal instrument includes a pair of micro-picks separated from each other about a central axis at a separation angle.

4. The ophthalmic surgical instrument of claim 1, wherein the membrane removal instrument includes at least one generally V-shaped micro-pick.

5. The ophthalmic surgical instrument of claim 4, wherein the membrane removal instrument includes at least one generally V-shaped micro-pick having a predetermined angle and a predetermined depth.

6. The ophthalmic surgical instrument of claim 1, wherein the membrane removal instrument includes a spatula configured for separation of the ophthalmic membrane from a retina.

7. The ophthalmic surgical instrument of claim 1, wherein the membrane removal instrument includes a spatula having a generally concave surface and an edge.

8. The ophthalmic surgical instrument of claim 1, wherein the membrane removal instrument includes a spatula having a tapered section.

9. A method of removing an ophthalmic membrane with a membrane removal instrument, comprising:
inserting the membrane removal instrument into an eye;
separating at least a portion of the ophthalmic membrane from a retina with the membrane removal instrument;
engaging at least a portion of the ophthalmic membrane with an aspiration port of the membrane removal instrument with a suction force; and
removing at least a portion of the ophthalmic membrane from the eye through an aspiration lumen of the membrane removal instrument with the suction force, the aspiration lumen of the membrane being in operative communication with the aspiration port.

10. The method of claim 9, further comprising, incising at least a portion of the ophthalmic membrane with a membrane removal instrument.

11. The method of claim 9, further comprising at least one micro-pick of the membrane removal instrument, the at least one micro-pick being configured for engagement with an ophthalmic membrane.
